(19) United States
(12) Patent Application Publication
(10) Pub. No.: US 2008/0015423 A1
(43) Pub. Date: Jan. 17, 2008

(54) Computer docking station for multiple health-related measurements

(76) Inventors: Phillip L. Lam, Monterey Park, CA (US); Ping Cheng Benjamin Liu, San Gabriel, CA (US)

Correspondence Address: LEONARD TACHNER, A PROFESSIONAL LAW;CORPORATION, 17961 SKY PARK CIRCLE, SUITE 38-E, IRVINE, CA, 92614, US

(21) Appl. No.: 11/823,526

(22) Filed: Jun. 27, 2007

Related U.S. Application Data
(62) Division of application No. 11/215,983, filed on Aug. 31, 2005, now Pat. No. 7,066,890.
(60) Provisional application No. 60/818,471, filed on Jul. 3, 2006.

(51) Int. Cl. A61B 5/00

(52) U.S. Cl. 600/301; 600/300; 600/309; 600/365; 600/368; 600/485

(57) ABSTRACT

A home health care kit in the form of a modularized computer docking station for allowing a user to provide self-monitoring test results that are transferred to a standard desktop or laptop computer for eventual automatic communication over the Internet to the user's physician. In a preferred embodiment, the invention comprises a plurality of interconnected test modules, each configured to provide everything needed to permit a user to conduct a health-related measurement. One such measurement may be that of blood pressure and pulse rate. Another may be actual blood sugar. Still another may be hemoglobin A1C or hemoglobin and another may be cholesterol and triglyceride levels. Each module has an electronics portion and a storage portion. Each such module also has a male connector on one side and a female connector on an opposing side so that the various modules may be interconnected, one to the next in sequence. The last module in a sequence of modules may then be connected in this fashion to a USB port of the user's desktop or laptop computer using either a USB cable or by means of a wireless interface adaptor.

### CROSS-RELATED APPLICATIONS

This application takes priority from U.S. Provisional Patent Application Ser. No. 60/818,471 filed Jul. 3, 2006 which takes priority from pending U.S. patent application Ser. No. 11/418,639 filed on May 5, 2006, which is a divisional of U.S. patent application Ser. No. 11/215,983 filed Aug. 31, 2005 and now issued U.S. Pat. No. 7,066,890.

### BACKGROUND OF THE INVENTION

1. Field of the Invention

The present invention relates generally to the field of health care. The invention relates more specifically to a computer docking station providing various health parameter measurements that can be conducted by a patient at home without direct medical supervision. The station, which can communicate with a desktop or laptop computer via USB interface or by wireless interface, comprises a plurality of measurement modules for measuring such parameters as blood pressure, pulse rate, blood sugar, hemoglobin A1C, and the like. The measurements are coordinated by the computer according to installed software and the measurement results are then transferred to the computer which may then communicate them via the Internet to a patient web site data center where they may be viewed by a patient's physician in due course.

2. Background Art

The methods for rendering routine medical care have undergone various changes over the past five or six decades. In the middle of the twentieth century, it was relatively common for general physicians to visit their patients at the patient's home. It was a common sight in the 1950's to observe a well-dressed doctor entering their patient's residence carrying their iconic little black leather bag of modest medical instruments. Eventually, the home visiting physician became a very rare sight except in the most rural communities. It became more and more commonplace for doctors to insist that their patients come to them and for those too sick to do that, to go or be taken to a hospital. In large measure even the individual physician's medical practice changed to where most medical treatment began to occur at large medical centers. Today, the majority of medical practice is carried out through large corporations such as HMO's and PPO's which hire physicians as salaried employees with well-organized supporting departments including on-site laboratories, x-ray facilities and even surgical centers.

One benefit of such evolutionary changes in the medical profession is that these large efficiently run and primarily profit-oriented medical enterprises have realized the benefit of preventative medicine as opposed to the earlier concept of merely treating sick people. They have discovered that medical corporations make more money in the long run if they devote a substantial effort to preventing illness by closely monitoring their member patients before their healthy conditions become chronic illnesses and before their chronic illnesses become acute and life threatening.

Such monitoring includes measurements relating to the most common illnesses of modern man, namely hypertension, diabetes and cardiovascular disease relating to high cholesterol and high triglyceride levels of the blood. Fortunately, today there are relatively new pharmaceutical treatments for these problems, but prescribing the proper dosages while avoiding negative side effects, requires careful and frequent monitoring of the patient. Such monitoring typically requires patients to make frequent visits to the medical facilities for laboratory tests and examinations by their physicians or physician's nurses or technical assistants. These frequent visits are expensive for the HMO or other type of medical corporation, expensive for the patient who must pay at least some form of co-payment therefore and of course, time consuming for both the patient and the medical personnel. Fortunately, the rise in preventative medical monitoring has seen concurrent improvements in medical instrumentation that is sufficiently sophisticated to enable patients to perform self-monitoring.

Two of the most prolific examples of improving self-monitoring instrumentation are in the areas of hypertension and diabetes. Today it has become common among the general public for non-medical, average individuals without any special training to monitor their own blood pressure, pulse rate and blood sugar. Companies such as HemoCue Inc. of Lake Forest, Calif. have also made it possible for individuals with little or no special training, to monitor their own hemoglobin A1C measurement (for extended average blood sugar), hemoglobin (for blood oxygen) and blood cholesterol. Another company by the name of Home Test Medical of Orlando, Fla., offers an over-the-counter product under the trademark “CardioChek Analyzer” which is a home testing instrument for measuring total cholesterol, HDL and LDL cholesterol, and triglyceride levels. Unfortunately, there is still a “disconnect” between a patient's ability to perform self-monitoring of key health parameters and a physician's or medical facility's willingness to rely on such measurements, yet alone to actually count on such measurements for making decisions in regard to patient treatment. It would be a significant step forward if there were an apparatus that both facilitated such self-monitoring measurements and provided a communication link for such measurements to be reviewed and utilized by physicians.

### SUMMARY OF THE INVENTION

The present invention is designed to facilitate two distinct functions required in enabling physicians to rely on health-related measurements taken by a patient or patient's family member at home. One such function is the actual performance of the measurement and the other such function is the transfer of the measurement results to the physician. In a preferred embodiment, the invention comprises a plurality of interconnected test modules, each configured to provide everything needed to permit a user to conduct a health-related measurement. One such measurement may be that of blood pressure and pulse rate. Another may be actual blood sugar. Still another may be hemoglobin A1C or hemoglobin and another may be cholesterol and triglyceride levels. Each module has an electronics portion and a storage portion. The electronics portion has the actual electronic and, if needed, optical components needed to perform the measurement. For example, the actual blood sugar module has an optical and digital electronics portion for receiving a test strip containing a drop of the user's blood and then passing a calibrated light through it to measure blood sugar based on detected light intensity. The storage portion of the actual blood sugar module has an enclosure containing vials of test strips and lancets for puncturing the fingertips to draw a drop of blood. Other modules may provide different devices in their respective storage portion. For example, the hemoglobin module may have a vial of microcuvettes each having a calibrated amount of ingredients designed to react in a predictable way to attenuate light based on the hemoglobin content of a drop of blood drawn in the microcuvette from the pricked finger of the user.

Each such module also has a male connector on one side and a female connector on an opposing side so that the various modules may be interconnected, one to the next in sequence. The last module in a sequence of modules may then be connected in this fashion to a USB port of the user's desktop or laptop computer using either a USB cable or by means of a wireless interface adaptor. The invention also comprises software in the form of an application program that is readily loaded into the desktop or laptop computer to which the modules are connected. This program is designed to provide instructions that may be displayed on the computer's monitor and which instruct the user as to when and how to conduct each measurement for which he or she has a corresponding module. The software also provides internal instructions that are compatible with the computer's operating system for receiving test measurement data from the modules, collecting the data in an appropriate buffer register and in an appropriate format, and eventually uploading the data via the Internet to a selected data center web address along with the user's name and other information, all of which may be encrypted for the user's privacy. That data may then be ultimately accessed by the user's physician or other enabled medical professional who has been pre-authorized to observe, download or otherwise utilize the user's measurement results.

Thus, it can be seen that the present invention comprises a home health care kit in the form of a modularized computer docking station for allowing a user to provide self-monitoring test results that are transferred to a standard desktop or laptop computer for eventual automatic communication over the Internet to the user's physician. Such a system will permit rather frequent monitoring of selected parameters of a person's health status at greatly reduced cost and time for both the patient and the medical personnel.

### BRIEF DESCRIPTION OF THE DRAWINGS

The aforementioned objects and advantages of the present invention, as well as additional objects and advantages thereof, will be more fully understood herein after as a result of a detailed description of a preferred embodiment when taken in conjunction with the following drawings in which:

FIG. 1 is a conceptual illustration of a system utilizing a preferred embodiment of the invention;

FIG. 2 is a perspective view of a typical blood pressure module of a preferred embodiment;

FIG. 3 is a perspective view of a typical blood sugar test module of a preferred embodiment; and

FIG. 4 is a perspective view of a typical lipid panel test module of a preferred embodiment of the invention.

### DETAILED DESCRIPTION OF A PREFERRED EMBODIMENT

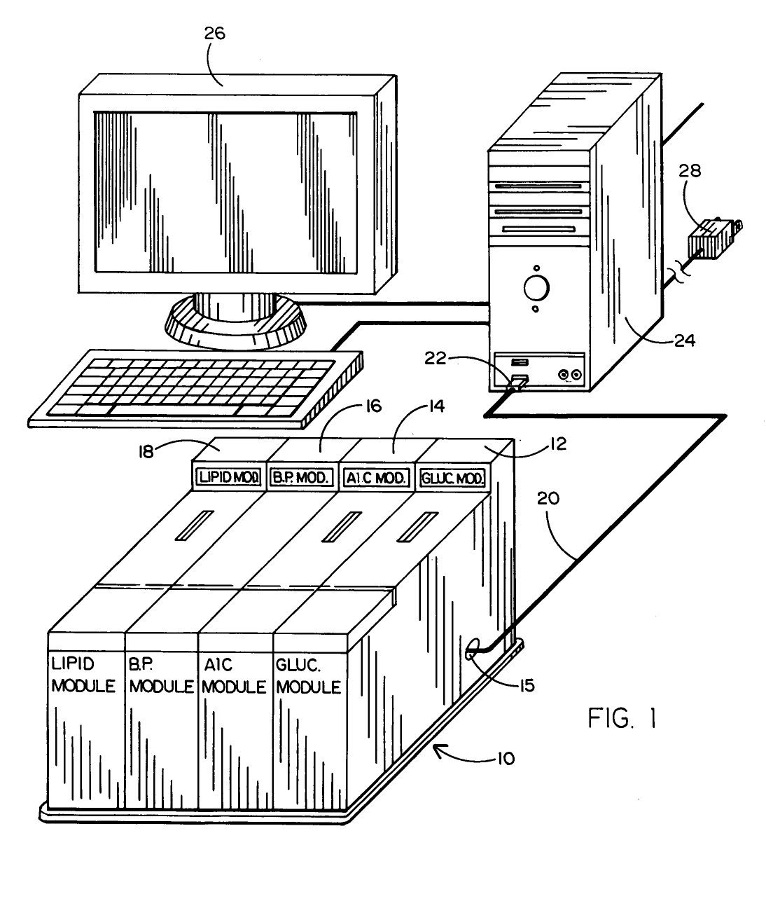

Referring to the accompanying drawings and to FIG. 1 in particular, it will be seen that a home health test station 10 comprises a plurality of contiguously arranged test modules 12,14,16 and 18 configured in proximity to a desk top computer 24, a computer monitor 26 and an Internet interface device 28. The various modules are preferably interconnected one to the next by female connectors 15 and their male counterparts (not shown) on opposing side surfaces of the respective modules. The end module in the sequence is connected to the computer chassis 24 by a suitable connector cable 20 which may, for example, be a USB connector cable which connects to the computer 24 at a USB port 22.

Each module 12, 14, 16, 18 provides all that is needed by a user to conduct a corresponding home health care test on himself or on a member of the household. By way of example, in the illustrated embodiment of FIG. 1, module 12 is a module for testing real time or current blood sugar, module 14 measures hemoglobin A1C, module 16 measures blood pressure and pulse rate and module 18 provides for testing lipid panel including total cholesterol, HDL and LDL cholesterol and triglycerides.

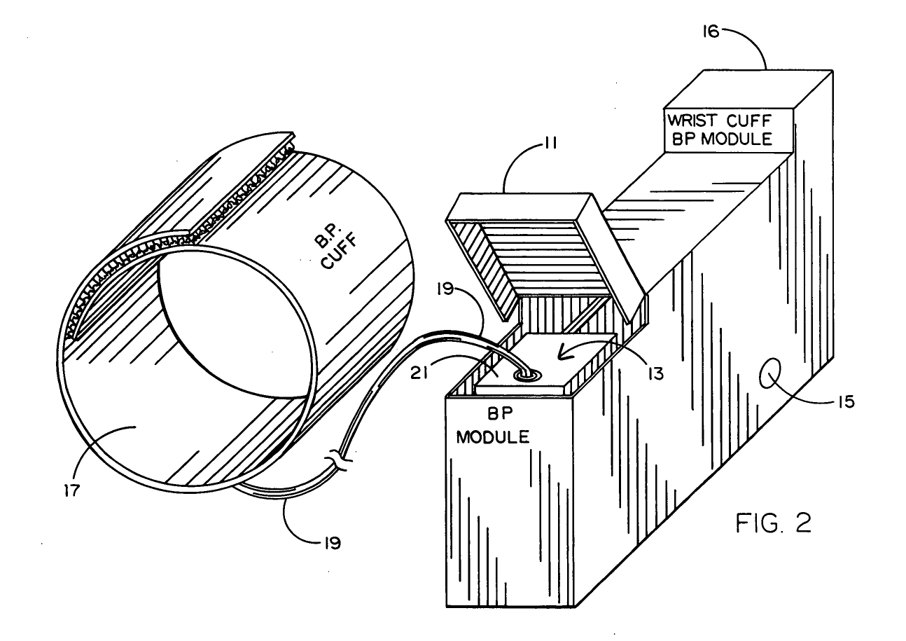

As seen in FIGS. 2 to 4, each such module provides an integral storage area for enclosing needed accessories associated with that particular measurement. As seen in FIG. 2, the blood pressure module 16 has a lid 11 enclosing a storage area 13 for enclosing a blood pressure cuff 17 attached by an elongated air tube 19 to an air pressure control device 21 which includes an air pump and pressure valve (not shown) which are well known components for conducting oscillometric testing of systolic and diastolic blood pressure as well as pulse rate.

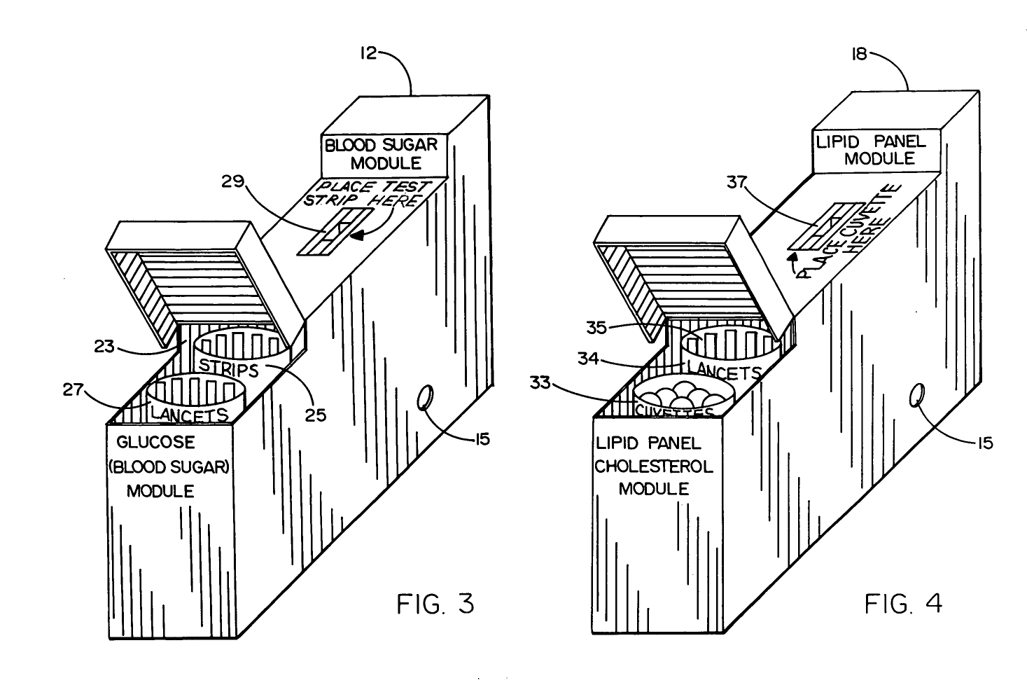

FIG. 3 illustrates a typical blood sugar testing module 12 in which the storage area 23 encloses a vial of test strips 25 and a vial of lancets 27. An optical detector 29 is provided on module 12 in which to place a test strip having a small drop of the user's blood as is well known for glucose test devices.

FIG. 4 illustrates a typical lipid panel-testing module 18 in which the storage area 31 encloses a vial of cuvettes 33 and a vial of lancets 35. An optical detector 37 is provided on module 18 in which to place the various cuvettes for reading lipid levels from a drop of the user's blood as is well known for lipid testers.

The computer 24 will have been programmed using a standard disc storage device to provide the user with procedural instructions and test schedule information based upon the computer's internal clock and/or instructions received over the Internet from the user's physician. Thus for example, after turning on his or her computer, the monitor 26 may display the message:
“Good morning Mr. Thomas, it's time for you to check your glucose level and your blood pressure. You should do the glucose test before consuming any food so your doctor can check your fasting blood sugar level. Please proceed with opening your glucose testing module and removing one test strip from the vial containing . . . . ”

After the user has followed the instructions on his or her monitor 24, a message may appear as follows:
“Thank you Mr. Thomas. You have successfully tested your blood sugar and your blood pressure. Your blood sugar results are 108 ml/l which is in the normal range for you after fasting at least 8 hours. Your blood pressure test was also successful. It showed a reading of 125/76 and a pulse rate of 68 ppm which are also normal for you after resting. These test results will be sent to your physician so that he can observe them and keep a record of them. Please note that on Friday, Jun. 30, 2006 you will be asked to check your hemoglobin A1C and your lipid levels. You will need to fast for at least 12 hours before testing your lipid levels. Thank you!”

The computer program will provide for categorizing and storing the user's test results and automatically encrypt them along with the user's identity so they can be uploaded via the Internet to a selected web site where the user's physician or other authorized personnel can access the data in due course.

It will now be understood that the present invention provides a unique and highly advantageous advance in health care by permitting average, untrained users to perform home-based tests of various key health-related parameters and automatically communicating the test results to their physicians. It will also be understood that the invention is not necessarily limited to the disclosed embodiments. By way of example, other health-related parameters may be suitable for such home-based modular configured testing such as EKG, blood oxygenation, body weight, body temperature and the like. Moreover, the present invention may also be configured as a plurality of entirely separate devices each communicating with a computer in the manner described. Thus, the scope hereof is not to be deemed limited by the disclosed embodiment, but only by the appended claims and their equivalents.

We claim:

1. A self-test health care apparatus for operation with a personal computer; the apparatus comprising:
a plurality of modules each configured for conducting a test of a corresponding health-related parameter and sending the resulting test data to said computer for monitoring and storage thereof.

2. The apparatus recited in claim 1 wherein at least one of said modules comprises a pressure cuff for measuring blood pressure.

3. The apparatus recited in claim 1 wherein at least one of said modules comprises a device for measuring blood glucose.

4. The apparatus recited in claim 1 wherein at least one of said modules comprises a device for measuring blood cholesterol.

5. The apparatus recited in claim 1 wherein at least one of said modules comprises a device for measuring blood hemoglobin.

6. The apparatus recited in claim 1 wherein said modules have compatible connections for docking to one another and have a common data bus for transferring test data to said personal computer.

7. The apparatus recited in claim 1 wherein said computer is programmed to remind users when to conduct at least one test of a health-related parameter.

8. The apparatus recited in claim 7 wherein said computer having at least one monitor for displaying messages relating to said health-related parameters and wherein said computer is programmed to utilize said monitor to remind users when to conduct said at least one test.

9. The apparatus recited in claim 1 wherein said computer is connected to the internet for sending test data of at least one health-related parameter to a physician authorized to receive such data.

10. The apparatus recited in claim 9 wherein said computer is programmed to encrypt said test data before sending it over the internet.
